(19) United States
(12) Patent Application Publication
(10) Pub. No.: US 2022/0280120 A1
(43) Pub. Date: Sep. 8, 2022

(54) SYSTEM FOR THE REMOTE ANALYSIS OF BIOMETRIC DATA RELATING TO PATIENTS WITH ONCOLOGICAL AND/OR ONCO- HEMATOLOGICAL DISEASES WITH COMORBIDITY AND/OR ADVERSE EVENTS

(71) Applicant: BBB S.R.L., Fisciano (IT)

(72) Inventor: Marco CITRO, Fisciano (IT)

(21) Appl. No.: 17/637,096

(22) PCT Filed: Aug. 27, 2020
(86) PCT No.: PCT/IB2020/057998

§ 371 Date: Feb. 22, 2022

(30) Foreign Application Priority Data
Aug. 27, 2019 (IT) 102019000015078

(51) Int. Cl. A61B 5/00; G16H 50/70; A61B 5/145; A61B 5/07; A61B 5/1477

(52) U.S. Cl. CPC A61B 5/7267; G16H 50/70; A61B 5/14503; A61B 5/14546; A61B 5/4839; A61B 5/073; A61B 5/1477; A61M 2037/0023

(57) ABSTRACT

A system for the remote analysis of biometric data relating to patients with oncological and/or onco-hematological diseases with comorbidity and/or adverse events, comprising one or more local monitoring infrastructures adapted to obtain biometric and/or diagnostic data relating to corresponding patients to be monitored, one or more communication networks each comprising one or more local communication devices adapted to receive data from a respective local infrastructure and one or more data processing units adapted to receive said data remotely from said local communication devices to define an IoT network, a centralized digital infrastructure adapted to receive data from said IoT networks for the generation of a database containing all the data detected by said local infrastructures and for the correlation thereof for storage in the cloud, a self-learning computational unit adapted to process said data stored in said centralized digital infrastructure.

### TECHNICAL FIELD

The present invention finds application in the field of data processing systems and particularly has as its object a system for the remote analysis of biometric data relating to patients with oncological and/or onco-haematological diseases with comorbidities and/or adverse events.

### STATE OF THE ART

As is known, the rapid development of technological tools and the increasing computing power available are increasingly stimulating the development of applications and technologies also in the medical field.

In particular, the current demographic forecasts see an increase in the average age in the population with an increase in oncological and onco-haematological diseases and related comorbidities.

This trend has led to a greater concentration of capital in the development of digital systems by the main pharmaceutical companies.

The area that has enjoyed the greatest technological developments is that relating to the analysis and management of comorbidities.

As matter of fact, the analysis of the manifestation of different pathologies for the same patient requires several medical-diagnostic devices and a higher computing power than expected for mono-pathological research, as well as unconventional analysis techniques, for example through Artificial Intelligence.

However, current approaches are mainly of the “Evidence Based” type, often relying on pivotal clinical trials for the use of new therapies wherein patients are highly selected excluding those of “Real Clinical Practice”.

It has been tried to overcome this limit through Real World Evidence (RWE) studies which consider all patients of daily clinical practice without distinction.

A new approach has made it possible to develop applications in the mono-pathological field, for example for diabetic patients, introducing the transition from a “Medicine Evidence Based” to a “Medicine Data Driven”, that is an approach that is based on the enormous collection of extemporaneous data on a large and real population of patients, as in an RWE, to process this data through Artificial Intelligence and Machine Learning in order to optimize the 360° clinical management of a patient, comprising therapeutic treatments and comorbidities, as well as related adverse events.

However, there is need for a Medicine Data Driven approach in oncology and onco-hematology.

As matter of fact, to date no systems have been developed in the oncology or onco-haematology field, i.e. in those sectors wherein there is a high number of tumor patients who have comorbidities of various kinds, including mainly cardiovascular and diabetes ones, with incidences increasing in proportion to the age of the patient.

### SCOPE OF THE INVENTION

The object of the present invention is to overcome the above drawbacks by providing a system for the remote analysis of biometric data relating to patients with oncological and/or onco-haematological diseases with comorbidities and/or adverse events adapted to collect a high number of extemporaneous data on a large and real population of patients for their processing to optimize the overall clinical management of the patient, including therapeutic treatments and comorbidities, as well as related adverse events.

A particular object is to provide a system for the remote analysis of biometric data relating to patients with oncological and/or onco-haematological diseases with comorbidities and/or adverse events that allows not only to continuously detect clinical data and clinical parameters from the patients but which also allows their “personalized processing” with the identification of the best therapy and a return to the patient with the automatic administration of drugs in relation to specific comorbiditics and adverse events.

Still another object of the present invention is to make available such a system that allows to perform the complex analysis of the data collected through the various elements of the system and historical data.

Still another object of the present invention is to provide such a system that allows a proactive interaction from and toward the patient through devices directly in contact with the patient, based on the correlation between the patient's lifestyle and personal reaction to the medical therapy.

Still another object of the present invention is to provide such a system which allows to verify the continuous improvement of the patient's nutrition by monitoring the effects of the applied therapy, for example a chemotherapy.

Not the least object of the present invention is to provide such a system that allows to achieve a strong cost reduction on the whole healthcare system and the optimization of the development of new therapies in the pharmaceutical industry.

These objects, as well as others that will become more apparent hereinafter, are obtained by a system for the remote analysis of biometric data relating to patients with oncological and/or onco-haematological diseases with comorbidities and/or adverse events which, according to claim 1, comprises one or more local monitoring infrastructures adapted to obtain biometric and/or diagnostic data relating to corresponding patients to be monitored, one or more communication networks each comprising one or more local communication devices adapted to receive data from a respective local infrastructure and one or more data processing units adapted to remotely receive said data from said local communication devices to define an IoT network, a centralized digital infrastructure able to receive data from said IoT networks for the generation of a database containing all the data collected by said local infrastructures and their correlation for storage in the cloud, a computational self-learning unit adapted to process said data stored in said centralized digital infrastructure.

Advantageous embodiments of the invention are obtained in accordance with the dependent claims.

### BRIEF DISCLOSURE OF THE DRAWINGS

Further features and advantages of the invention will be more apparent in the light of the detailed description of preferred hut not exclusive embodiment of the system according to the invention, illustrated by way of non-limiting example with the aid of the accompanying drawing tables wherein:

FIG. 1 is a diagram wherein the system architecture is schematized;

FIG. 2 is a communication scheme between the various elements of the system.

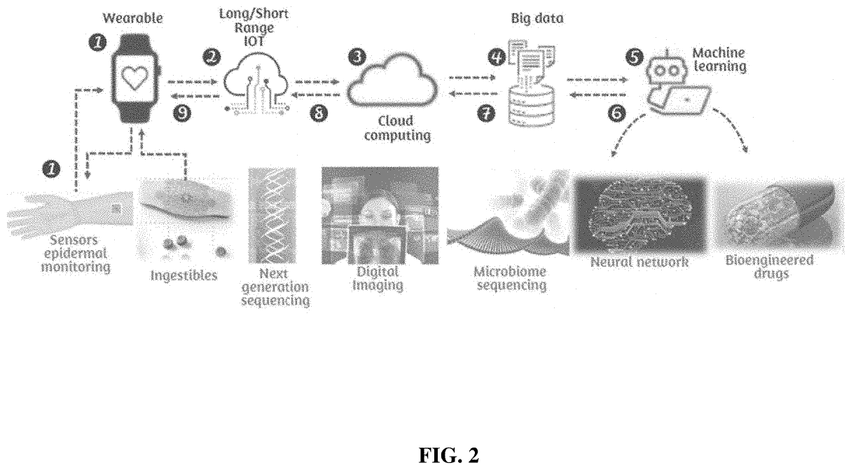

### BEST MODES OF CARRYING OUT THE INVENTION

With reference to the attached figures, a possible architecture is shown for a system for the remote analysis of biometric data relating to patients with oncological and/or onco-haematological diseases with comorbidities and/or adverse events, in particular oncological or onto-haematological patients with cardiovascular-metabolic comorbidities and/or Adverse Events (AE).

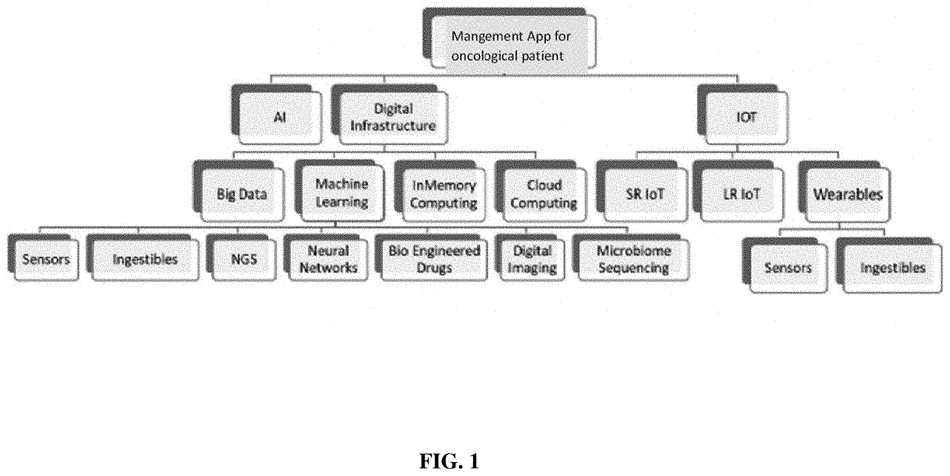

In its most essential scheme of FIG. 1, the system comprises one or more local monitoring infrastructures suitable for obtaining biometric and/or diagnostic data relating to corresponding patients to be monitored, one or more communication networks each comprising one or more communication local devices adapted to receive data from a respective local infrastructure and one or more data processing units adapted to remotely receive such data from local communication devices, so as to define an IoT (Internet of Things) network, a centralized digital infrastructure adapted to receive data from the IoT networks for the generation of a database containing all the data detected by local infrastructures and their correlation for storage in the cloud, a self-learning computational unit adapted to process the data stored in the centralized digital infrastructure through machine operations learning.

Each local infrastructure will be associated with a single patient and will consist of a multiplicity of devices designed to detect clinical data and/or biometric parameters and communicate with each other wirelessly, for example via Bluetooth® protocol, to create a Short-Range IoT network (SR IoT).

In particular, a local infrastructure will comprise one or more wearable type monitoring devices to come into contact with the respective patient to be monitored and to acquire biometric parameters to be sent to a local communication device owned by the patient. In turn, the various communication devices will be adapted to communicate at long range with the processing unit to define an additional Long-Range IoT network (LR IoT).

According to a preferred but not exclusive embodiment, the local communication devices may be selected between smartphones, tablets, phablets, notebooks or other mobile communication devices on which will reside a specific software application programmed to communicate with the centralized digital infrastructure.

In an exemplary and non-limiting manner, the wearable device may be a smartwatch or other device adapted to detect biometric parameters, for example by monitoring the heart rate with relative extemporaneous electrocardiogram, monitoring the circadian rhythm, monitoring sleep and the like, or it may be a Graphene bracelet for blood glucose monitoring, such as those already developed and in use for diabetic patients. Another type of monitoring device could be an ingestible sensor such as a pill with a sensor with a square millimeter extension, adapted to be ingested by the patient to be activated by the contact of the electrolytes present in the patient's body in order to obtain information on the health thereof.

This information will then be sent wirelessly from the sensor, for example always via Bluetooth® protocol, to the corresponding local communication device and from this to the clinician and the digital infrastructure, to then be processed in machine learning. The advantage in the use of this technology is represented by the possibility of providing continuous monitoring of the patient and allowing the verification of compliance with oral therapy, for example in cases of chronic myeloid leukaemia (CML), cardiac comorbidities and the like, and the efficacy thereof, which can be assessed in the case of CML with the blood BCR/ABL transcript value and in the case of cardiac comorbidities by monitoring the values of parameters such as heart rate and blood pressure.

Furthermore, it is possible to use skin monitoring sensors provided with a layer of graphene adapted to adhere to the patient's skin to detect changes in one or more biochemical parameters and send the relative information to the corresponding local communication device.

For example, these sensors could be similar to thin bracelets that adhere to the skin and thanks to the presence of a layer of graphene they will have particular electronic properties that will allow to detect changes in biochemical parameters such as glucose, pH, humidity, temperature, heart rate, blood pressure. and similar.

The biochemical parameters detected may be managed directly on the display of the local communication device, such as a smartphone, or sent to the centralized digital infrastructure for data analysis and processing.

These wrist sensors may also be equipped with micro-needles that graft the skin and release the amount of drug, as needed for the management of the disease, more expressed EA and comorbidity.

In this way it will be possible to realize real targeted therapies (target therapy), in which the pharmacodynamics and pharmacokinetics of a drug may be evaluated patient by patient through secondary parameters managed with machine learning, in order to allow the optimization of the treatment, with consequent positive impacts also from an economic and cost saving point of view.

Each of the local infrastructures may also be implemented with Digital Imaging devices for the correlation of diagnostic images, such as MRI, CT, ultrasound scans and the like, with the clinical parameters of the patient, for the purpose of their joint analysis through machine learning and comparison with an already available dataset that increases more and more and allows to continuously refine the analysis, whose output (clinical insights) is provided in real time to the clinician for optimal patient management.

Additional devices for the acquisition of clinical information could be genetic sequencing devices or the patient's DNA tracts, or the so-called NGS (Next Generation Sequencing).

The resulting advantage is the rapid sequencing of genes or stretches of DNA to detect any point mutations potentially underlying the disease.

Furthermore, it will be possible to operate using Microbiome Sequencing to find information on the intestinal microbiota, essential for patient survival as it allows the continuous improvement of the patient's nutrition by monitoring the effects of chemotherapy.

As matter of fact, many metabolites and biomolecules produced by intestinal microbes are essential for the correct functional performance of the patient, but chemotherapy therapies may often destroy part or all of the patient's intestinal microbiota and therefore its continuous monitoring is essential for its integration, when necessary. Data obtained by Microbiome Sequencing may also be analysed in machine learning. The centralized digital infrastructure, also provided with in-memory data processing computational unit to accelerate data access speeds, will thus be able to generate a complex set of data (Big Data) from various wearable devices or devices adapted to come into contact with the body of the patient, as well as through Digital Imaging operations and genetic analysis (NGS).

In addition, the digital infrastructure will receive all the patient's clinical parameters, such as heart rate, glycemia, biochemical parameters and the like, for their storage in the cloud.

This solution for storing information has the advantage of allowing access to all research bodies participating in the project so that all data can be constantly updated. The so collected data will be made available for the subsequent treatment and analysis step and to be used through management solutions of therapies/Adverse Events/comorbidities customized for each patient.

The collection of data, both historical and from the devices worn by patients, will make it possible to create a huge information bank and the starting database that feeds the Machine Learning for the subsequent analysis and processing step.

From above, it is clear the double advantage of cloud storage, first of all for the individual patient and then for the patient population in general: more specifically it is possible to manage patients not only through the analysis of data in their entirety but also through a subdivision by gender (distinction of sex between patients) which can provide a further refinement of targeting between patients of different sexes, as men and women may respond differently to the same drug treatment.

The heart of the system will be the self-learning computational unit based on Machine Learning and which allows to process a large amount of data and manage the correlation between Pathologies/Adverse Events/Comorbidities or identify new ones if not known.

The self-learning computational unit makes it possible to remotely manage pathologies, comorbidities, adverse events through a data path toward and from patients and allows a real targeting of therapy for each patient.

Machine Learning increases the predictive capabilities to improve the management of therapeutic treatments/Adverse Events/Comorbidities to define personalized therapeutic treatments for the various patients.

An important element for the self-learning unit will be an artificial neural network (Neural Network) that allows to calculate different treatment scenarios as the patient's clinical parameters change.

The information output of Machine Learning may also be exploited to generate new bioengineered drugs, such as monoclonal, bispecific antibodies, CAR-T, Oncoviruses DNA and the like.

The system according to the invention is susceptible of numerous modifications and variations, all falling within the inventive concept expressed in the attached claims.

All the details may be replaced by other technically equivalent elements, and the materials and tools may be different according to the needs, without departing from the scope of protection of the present invention.

1. A system for the remote analysis of biometric data relating to patients with oncological and/or onco-hematological diseases with comorbidity and/or adverse events, comprising:
one or more local monitoring infrastructures adapted to obtain biometric and/or diagnostic data relating to corresponding patients to be monitored;
one or more communication networks each comprising one or more local communication devices adapted to receive data from a respective local infrastructure and one or more data processing units adapted to receive said data remotely from said local communication devices to define an IoT network;
a centralized digital infrastructure adapted to receive data from said IoT networks for the generation of a database containing all the data detected by said local infrastructures and for the correlation thereof for storage in the cloud;
a self-learning computational unit adapted to process said data stored in said centralized digital infrastructure.

2. System as claimed in claim 1, characterized in that each of said local infrastructures comprises one or more monitoring devices suitable for coming into contact with the respective patient to be monitored for the acquisition of biometric parameters.

3. System as claimed in claim 2, characterized in that one or more of said monitoring devices are adapted to be worn by the patient to detect one or more biometric parameters and to send said information wirelessly to one of said local communication devices.

4. System as claimed in claim 3, characterized in that one or more of said monitoring devices are ingestible sensors adapted to be activated by the contact of the electrolytes present in the patient's body to obtain information on the health thereof and to send wirelessly said information to one of said local communication devices.

5. System as claimed in claim 4, characterized in that one or more of said monitoring devices are monitoring skin sensors provided with a graphene layer adapted to adhere to the patient's skin to detect variations in one or more biochemical parameters and sending the related information to one of said local communication devices.

6. System as claimed in claim 5, characterized in that said skin monitoring sensors are provided with micro-needles which engage the skin for the release of a drug.

7. System as claimed in claim 1, characterized in that each of said local infrastructures comprises one or more Digital Imaging devices for the correlation of diagnostic images, such as MRI, CT scans, ultrasound scans and the like, with the clinical parameters of the patient.

8. System as claimed in claim 7, characterized in that each of said local infrastructures comprises one or more devices for genetic sequencing or DNA stretches of the patient.

9. System as claimed in claim 8, characterized in that said centralized digital infrastructure comprises a computational unit for in-memory data processing.

10. System as claimed in claim 1, characterized in that said self-learning computational unit comprises an artificial neural network.
